(19) United States
(12) Patent Application Publication
(10) Pub. No.: US 2024/0226559 A9
(43) Pub. Date: Jul. 11, 2024

(54) Cure for Bell's palsy and Ramsay Hunt syndrome

(71) Applicant: Anuthep Benja-Athon, New York, NY (US)

(72) Inventor: Anuthep Benja-Athon, New York, NY (US)

(21) Appl. No.: 17/973,513

(22) Filed: Oct. 25, 2022

(51) Int. Cl. A61N 1/36; A61N 1/05; G16H 20/70

(52) U.S. Cl. CPC A61N 1/36067; A61N 1/0526; A61N 1/36003; A61N 1/36132; G16H 20/70; A61N 1/37247

(57) ABSTRACT

The cure for Bell's palsy and Ramsay Hunt syndrome comprised individual series of electrical stimulus for relieving the compression of ipsilateral facial nerve in its facial canal. The compositions of selfies and related avatars of patients recommending frequencies, durations and numbers of individual treatment sessions for achieving full recovery and digital blockchains managing the cure among patients.

This application is a continuation-in-part of U.S. Pat. No. 5,861,015 entitled the Modulation Of The Nervous System For Treatment Of Pain And Related Disorders filed 1999 Jan. 19.

### FIELD OF THE INVENTION

The cure for Bell's palsy and Ramsay Hunt syndrome

### BACKGROUND OF THE INVENTION

Bell's palsy is a medical emergency (J. A Hilger 1965 and L. B. W. Jongkees 1972). Every 13 minutes one American comes down with Bell's palsy. (K. K. Adour 1972) This facial disfiguring disease of epic proportion has an incidence of 23 per 100,000 people per year. (W. A. Hauser 1971). This figure translates to a staggering number of 67,000 Americans per year.

In Bell's palsy, the facial canal is the site in which the facial nerve is compressed and damaged. (F. H. McGovern et. al., 1961 and L. B. W. Jongkees 1972) An evidence supporting the facial canal as the origin of Bell's palsy is a report that there were no relapses in patients with relapsing facial palsy after facial canal were surgically opened. (K. Kettel 1947)

This should be obvious to physicians dealing with the compression of a vital nerve. Bell's palsy is an epic-proportion catastrophe for any men, women and children. It is accurate to state that every adult knows of or has seen someone with the life-long facial disfigurements and associated facial dysfunctions and numerous sequelae including surgeries, daily pain and suffering, life and family disruptions and immense tangible losses of livelihoods caused by Bell's palsy. So, why the prevalent—albeit wrong—attitude and advices that Bell's palsy is an affliction that spontaneously resolved in 11 patients? One of the vital reasons heretofore is no effective treatment and misinformation.

According to the early literature (reliability cannot be verified):

| | SPONTANEOUS RECOVERY RATE |
|---|---|
| General accepted | 75%-90% |
| One report - the prognosis for | 65% (W. B. Matthews 1961) |
| complete recovery | |
| Satisfactory return of function | 55% (J. A. Hilger 1965) |
| Satisfactory return of function | 50% (T. Cawthorne 1950) |

Just as bad, Bell's palsy and Ramsay Hunt syndrome patients and families et al. are helplessly afflicted with immense emotional, psychological and traumatic sequelae.

For historical record, Bell's palsy et al., in 2022, heretofore remain an enigma for 7+ billions people including all physicians except this pro se applicant.

U.S. Pat. No. 5,861,015 1999 of 1999-01-19 has the scientific and medical facts serving as the foundation for enabling 2001 Bell's Palsy eBook of 2001 based on 36 Bell's palsy and Ramsay Hunt syndrome patients. The present invention is based on about 250 Bell's palsy and Ramsay Hunt syndrome patients and the wealth of information not present before 2001.

The prerequisite for this invention is the unique combination—knowledge and fortitude of a physician scientist with accesses to and skill in human anatomy, neuroanatomy, neurophysiology including neurogenic inflammation*—unbeknownst to most physicians—neurology, medicine, acupuncture science, surgical skill, hundreds of Bell's palsy and Ramsay Hunt syndrome patients, referring physicians and patients—and the precise alignment of the aforementioned.

In summary, the only cure for Bell's palsy and Ramsay Hunt syndrome and solution to aforementioned matters known to mankind.

Benja-Athon, A. 2001 Bell's Palsy. Cause, Treatment and Prevention™ provides data and information. This is an eBook at Amazon.com.

### SUMMARY OF THE INVENTION

The cure for Bell's palsy and Ramsay Hunt syndrome heretofore is only known to this physician applicant. This invention benefits patients and physicians all over the world for eternity.

The cure for individual patients, use an anode electrode and a cathode electrode which are percutaneously implanted so that the tip and adjacent portion of the shaft are reversibly implanted in ipsilateral masseter and ipsilateral sternocleidomastoid. Individual series of individual electrical stimulus modulating neuronal activities in said body parts and related neuronal circuitries thereby relieving the compression of ipsilateral facial nerve in its facial canal. At least two digital platforms rendering compositions of smartphones-rendered and videocam-rendered serial BP starts of treatments-full recoveries selfies and related avatars in said patients and managing said treatments and financial services related to the said treatments.

Said anode electrode and cathode electrode for percutaneously conducting and delivering said electrical current for modulating said neuronal circuitries and activities of peripheral and cranial nerves and central nervous system.

### DESCRIPTIONS OF THE PREFERRED EMBODIMENTS

As a continuation-in-part of U.S. Pat. No. 5,861,015, the following is the innovative scientific invention based on 2001—the present invention:

The medical treatment thus cure for Bell's palsy and Ramsay Hunt syndrome, in individual patients, use an anode needle electrode and a cathode needle electrode, which are reversibly and percutaneously inserted through the facial skin and neck skin, respectively, of said individual patients. Said anode needle electrode is percutaneously implanted so that the tip and adjacent portion of the shaft of said anode electrode are in the masseter. Said cathode needle electrode is percutaneously implanted so that the tip and adjacent portion of the shaft of said cathode electrode into or adjacent to the tendomuscular junctions and muscles—close to or adjacent to the afferent neurons—of ipsilateral sternocleidomastoid inserting into the mastoid process (Hollinshead, W H. Textbook of Anatomy 2nd ed, Harper & Row 1967, 1962).

In U.S. Pat. No. 5,861,015, the standard and readily available monopolar acupuncture pin and needle are used with range of lengths of about 20-125 millimeters and a range of cross-sectional diameters of 0.1 mm-0.71 mm. The excitation and inhibition of said nerves and nerve receptors are achieved by passing 1-500 hertz direct electrical current from a common commercially available current generating device via electrical leads to said electrodes to said nerves and nerve receptors innervating said effector.

In said 250 Bell's palsy and Ramsay Hunt syndrome patients, said monopolar acupuncture anode and cathode needles have diameter of 0.16 millimeter×length of 30 millimeters.

This applicant used—for said 250 patients—the stimulation set at frequency 6 Hz and intensity low 6 (the device being used for the patients has the ranges of frequency 2-10 Hz, intensity low 1-20, medium 20-100, high 100-500). Patient should be able to perceive said action potential generation by the sensation perceived. At the onset of the stimulation, every patient was asked to be stimulated with strong albeit no pain and comfortable stimulation. Said application of said current stimulus is approximately 12 minutes.

Said disposable electrodes are reversibly removed and discarded after 12 minutes of said treatment. All electrodes are sterile and single-use disposable.

Tearings resolved within 24 hours after the first treatment session in the BP eye in all patients except one patient who took a few days.

In said patients, the average days from the onset of treatment to full recovery=12 days. Full recovery is defined as individual treated patients have or approximately symmetrical tone and voluntary movements of bilateral foreheads, earbrows, eye closures, cheeks and lips. However, only patients can feel the residual weakness, albeit sooner resolved, of the treated face.

In other words, said average days from the onset of treatment to full recovery=12 days wherein the numbers of individual treatment sessions in individual patients=f(frequencies and intervals between treatments thus dates and durations thus numbers of individual treatment sessions). The range of numbers of individual treatment sessions is approximately a few days—several weeks from the onset of treatment to full recovery due to various biological and temporal factors in patients.

Similarly, as in U.S. Pat. No. 5,861,015, the use of a concentric metallic needle electrode close or adjacent to said tendomuscular junction and muscle spindles of ipsilateral sternocleidomastoid.

Similarly, the use of a concentric metallic needle electrode close or adjacent to said tendomuscular junctions and muscle spindles of ipsilateral sternocleidomastoid.

Similarly, as in U.S. Pat. No. 5,861,015, any common commercially available transcutaneous electromagnetic stimulator wherein anode electrode and cathode pad producing transcutaneous electromagnetic stimulus can be experimented. Said anode electrode pad is attached to the skin anterior to the earlobe and of said anode electrode are in the masseter. Said cathode needle electrode is percutaneouly implanted so that the tip and adjacent portion of the shaft of said cathode pad is attached to the skin superficial to the tendomuscular junction of the origin of ipsilateral sternocleidomastoid. Said transcutaneous electromagnetic stimulus is strong albeit no pain and comfortable stimulation.

Individual clocks for displaying the durations of the delivery of said electrical current. Said application of said stimulus is approximately 12 minutes.

Never before in the history of mankind, first, the medical treatment thus cure for Bell's palsy and Ramsay Hunt syndrome comprise networked repositories-rendered expandable interactive and expandable compositions networked repositories-rendered expandable interactive and expandable compositions of smartphones-rendered and videocam-rendered serial BP onsets-sarts of treatments-full recoveries selfies and related avatars, said networked repositories-rendered expandable interactive and expandable compositions uniquely and instantly rendering, for all patients and physicians, using their networked communications devices, in USA and other nations on earth:
first unique innovation, the optimal and most efficient and cost-effective digital platform for delivering and applying said medical treatment thus cure; and
second unique innovation, abiotic-intelligent said networked repositories-rendered interactive and adaptable trends, prognostications and time courses and rates in and among said patients achieving full recovery, wherein compositions for recommending frequencies and intervals, dates, durations and numbers of individual treatment sessions for achieving full recovery in individual patients.

Individual BP patients use their networked smartphones, laptops, desktops or videocam to take individual series of selfies and videocam-rendered serial BP onsets-sarts of treatments-full recoveries selfies, which attached patients' demographics and personal data, which are transmitted online to servers. Servers take said transmissions and compile said selfies and related avatars based on the information, data and their derivatives present herein Description and Claims to render said networked repositories-rendered expandable interactive and expandable compositions.

Furthermore, said networked repositories can use commercial software to convert individual series of selfies and videocam-rendered serial BP onsets-starts of treatments-full recoveries selfies and said compositions of said individual series of selfies and videocam-rendered serial BP onsets-starts of treatments-full recoveries selfies into compositions of said individual series of selfies and videocam-rendered serial BP onsets-starts of treatments-full recoveries avatars based on the information, data and their derivatives present herein Description and Claims.

Said selfies and related avatars will render progresses of facial tone, expressions and muscular contractions of frontal, orbicularis oculi, naris, cheeks, lips and chins. Tearings, tastes and speeches will be included.

For example, said networked repositories comprised mathematical and stat programs for compiling analyzed and rendered said information and data in said selfies and related avatars to rendering interactive said networked repositories-rendered expandable interactive and expandable compositions of mathematical and statistical data, such as, but not limited, to ranges, averages, modes and medians with standard deviations, on frequencies, durations and numbers of individual treatment sessions for achieving full recovery in all patients for patients and physicians. Said compositions of grades and ranges of facial tones, facial muscle contractions and degrees of symmetries of various regions of faces.

In summary, said first unique innovation coupled with second unique innovation provide said mathematical and statistical data for any and all patients and their families and concerned and physicians heretofore instantly engage, understand and comply, albeit all patients did, with the medical treatment thus cure while most efficiently cope, deal with and eliminate said immense emotional, psychological and traumatic enigma and sequelae.

Second, the medical treatment thus cure for Bell's palsy and Ramsay Hunt syndrome comprise digital blockchains for managing said treatments such as, but not limited to, that the cure will be known to people who can instantly introduce patients to the cure which is optimally rendered anywhere on earth. The financial services related to the said treatments among all parties, in and among viewing and touchscreens, in and among networked communication devices of said patients, physicians and payors such as, but not limited to, healthcare insurers or other 3rd parties, of said cure.

Although various preferred embodiments of this invention have been described, it will be appreciated by those skilled in the art that variations and adaptations may be made without departing from the spirit of the invention or the scope of the aforementioned claims.

Although the preferred techniques, stimuli, and instruments have been described in this application, it will be appreciated by those skilled in the art variations and adaptations may be made to said modes without departing from the spirit of the invention or the scope of the aforementioned claims.

Although various preferred embodiments of this invention have been described in human, it will be appreciated by those skilled in the art that its application and use can be applied to other tunnel diseases such as, but not limited to, carpal tunnel syndrome, tarsal tunnel syndrome and other tunnel diseases and in other living species without departing from the spirit of the invention or the scope of the claims.

1. The activated switch for rendering electrical circuitries for curing Bell's palsy and Ramsay Hunt syndrome collectively known herein as BP in patients, wherein said activated switch-rendered electrical circuitries for relieving the compression of ipsilateral facial nerve in its facial canal, and wherein electrodes for conducting and delivering individual series of individual electrical stimulus for activating and modulating said activated switch-rendered electrical circuitries in individual patients, said individual series for relieving the compression of ipsilateral facial nerve in its facial canal, and networked repositories-rendered expandable interactive and expandable compositions of smartphones-rendered and videocam-rendered serial BP starts of treatments-full recoveries selfies and related avatars in said patients, said interactive and expandable compositions comprising networked repositories-rendered interactive and adaptable trends, prognostications, time courses and rates of individual facial responses for recommending frequencies, durations and numbers of individual treatment sessions for achieving full recovery and rendering emotional, psychological therapeutics and guidances in individual patients and relatives, and digital blockchains for achieving and managing said medical cure among said patients, physicians and payors of said cure, in and among viewing and touchscreens, in and among networked communication devices, said cure comprise:
commercial electricity generators for generating electricity, leads for conducting said electrical current, and anode electrode and cathode electrode for conducting and delivering said electrical current for activating said switch; and
said networked communication devices comprised networked smartphones, laptops, desktops and servers comprised said networked repositories and said touchscreens and viewing screens, and cameras and microphones.

2. The activated switch according to claim 1 comprise ipsilateral sternocleidomastoid inserting into the mastoid and surrounding soft tissues in and of ipsilateral facial canal ipsilateral masseter;
the afferent neurons innervating ipsilateral sternocleidomastoid inserting into the mastoid, afferent neurons innervating the surrounding soft tissues, ipsilateral masseter and the ipsilateral facial canal;
said anode electrode and said cathode electrode for conducting and delivering electrical current for activating said switch; and
commercial electricity generators for connecting said leads for conducting said electrical current to said anode electrode and cathode electrode for conducting and delivering said electrical current to said anode electrode and said cathode electrode.

3. The afferent neurons innervating ipsilateral sternocleidomastoid according to claim 2 comprise:
afferent neurons innervating the tendon organs of said sternocleidomastoid; and
afferent neurons innervating the muscle spindles of said sternocleidomastoid.

4. The activated switch-rendered electrical circuitries according to claim 1 wherein said electrical circuitries comprise:
said activated switch;
said afferent neurons innervating ipsilateral sternocleidomastoid, and afferent neurons innervating surrounding soft tissues, ipsilateral masseter and soft tissues surrounding and in ipsilateral facial canal;
sympathetic and parasympathetic neurons innervating ipsilateral facial canal and surrounding soft tissues; and
ascending and descending neuronal tracts for relaying said connections-rendered electrical signals, in the central nervous system.

5. The medical cure for BP in patients, comprising certain facial and ipsilateral neck body parts where electrodes for conducting and delivering individual series of individual electrical stimulus for modulating neuronal activities in said body parts and related neuronal circuitries in individual patients, said series of modulations for relieving the compression of ipsilateral facial nerve in its facial canal, and networked repositories-rendered expandable interactive and expandable compositions of smartphones-rendered and videocam-rendered serial BP starts of treatments-full recoveries selfies and related avatars in said patients, said interactive and expandable compositions comprising networked repositories-rendered interactive and adaptable trends, prognostications, time courses and rates of individual facial responses for recommending frequencies, durations and numbers of individual treatment sessions for achieving full recovery and rendering emotional, psychological therapeutics and guidances in individual patients and relatives, and digital blockchains for achieving and managing said medical cure among said patients, physicians and payors of said cure, in and among viewing and touchscreens, in and among networked communication devices, said cure comprise:
commercial electricity generators for generating electricity, leads for conducting said electrical current, and anode electrode and cathode electrode for conducting and delivering said electrical current for activating said switch; and
said networked communication devices comprised networked smartphones, laptops, desktops and servers comprised said networked repositories and said touchscreens and viewing screens, and cameras and microphones.

6. The medical cure for BP in patients according to claim 5 wherein said individual electrical stimulus for modulating neuronal activities in said body parts and related neuronal circuitries in individual patients comprise said anode electrode and said cathode electrode for conducting and delivering electrical current for activating and modulating neuronal activities of the afferent neurons innervating ipsilateral sternocleidomastoid inserting into the mastoid, surrounding soft tissues in and around ipsilateral facial canal, and ipsilateral masseter.

7. The afferent neurons innervating ipsilateral sternocleidomastoid according to claim 6 comprise;
afferent neurons innervating the tendon organs of said sternocleidomastoid; and
afferent neurons innervating the muscle spindles of said sternocleidomastoid.

8. In the cure for Bell's palsy and Ramsay Hunt syndrome in patients, the method of curing BP in individual patients, comprising certain facial and ipsilateral neck body parts where electrodes for conducting and delivering individual series of individual electrical stimulus for modulating neuronal activities in said body parts and related peripheral nerves and pathway in the central nervous system in individual patients, said series of modulations relieving the compression of ipsilateral facial nerve in its facial canal, and, first, networked repositories rendering expandable interactive and expandable compositions of smartphones-rendered and videocam-rendered serial BP starts of treatments-full recoveries selfies and related avatars in said patients, said interactive and expandable compositions rendering interactive and adaptable trends, prognostications, time courses and rates of individual facial responses which recommending frequencies, durations and numbers of individual treatment sessions to achieving full recovery and rendering emotional, psychological therapeutics and guidances in individual patients and relatives, and, second, digital blockchains achieving and managing said medical cure among said patients, physicians and payors of said cure, in and among viewing and touchscreens, in and among networked communication devices, said method comprised the processes of:
commercial electricity generators generating electricity, to which connected leads for conducting said electrical current to said anode electrode and cathode electrode conducting and delivering said electrical current activating and modulating neuronal activities in said body parts and related somatic nerves, autonomic nerves and ascending and descending pathways in the central nervous system in individual patients; and
said individual of series of modulations relieving the compression of ipsilateral facial nerve in its facial canal in individual patients.
